(19) United States
(12) Patent Application Publication
(10) Pub. No.: US 2022/0409473 A1
(43) Pub. Date: Dec. 29, 2022

(54) A CORPOREAL COMPRESSION SYSTEM

(71) Applicant: Coco Industries Pty Ltd, Swanbourne (AU)

(72) Inventor: Christopher MITCHELL, Swanbourne (AU)

(21) Appl. No.: 17/775,665

(22) PCT Filed: Nov. 11, 2019
(86) PCT No.: PCT/AU2019/051241

§ 371 Date: May 10, 2022

(51) Int. Cl. A61H 9/00

(52) U.S. Cl. CPC A61H 9/0021; A61H 2009/0035; A61H 2201/0103; A61H 2201/0142; A61H 2201/5087; A61H 2201/5071; A61H 2203/0456

(57) ABSTRACT

A corporeal compression system for use in applying compression to a patient positioned upon a supporting surface has an overlay, and a gas supply system. The overlay includes flexible sheet material that is arranged into at least two layers to define an internal region therebetween, an inlet orifice that opens into the internal region, and restraints that, in use, locate and restrain edge portions of the flexible sheet material. The layers are repositionable with respect to each other such that the sheet material can assume a deflated state in which the volume of the internal region is a minimum In use of the corporeal compression system, the flexible sheet material of the overlay is draped over the patient, the flexible sheet material is restrained relative to the support surface by the restraints, and the gas supply system is operable to deliver gas to increase the volume of the internal region from the deflated state and establish an elevated pressure within the internal region, thereby compressing the patient between the posterior layer of the overlay and the supporting surface.

### FIELD OF THE INVENTION

The present invention relates to a corporeal compression system. More particularly, the present invention relates to a system for use in applying compression to a patient for management and/or assistance in medical interventions or investigations.

### BACKGROUND

There are instances in which it is desirable to apply compression for management and/or assistance in medical interventions or investigations. Compression can be advantageous in managing internal fluid pressure or distribution within a patient's body. Further, compression can be advantageous in partial immobilisation of a patient in order to limit discomfort and/or prevent exacerbation of an existing condition.

As an example of managing blood pressure of a person or animal (hereinafter referred to as a “patient”), it may be desirable to increase a patient's venous blood pressure in response to an adverse indication, or as a part of an investigative or surgical procedure. It is known that a decrease in venous blood pressure is detrimental to the ability of the heart to move blood into the arterial side of the cardiovascular system. This is because cardiac output is directly linked to venous return, which is partly determined by the venous blood pressure.

In many occurrences, a decrease of venous blood pressure can be attributed to loss of blood volume (by internal bleeding or external bleeding), and/or venodilation.

Venodilation can be caused by many conditions, including sepsis, anaphylaxis, spinal injury, and drug interactions.

Currently known pharmacological treatments of acute, severe venodilation are limited and have a low efficacy. For example, in the case of anaphylaxis, administration of adrenaline can increase peripheral vascular resistance and cardiac function but does not increase venous tone. Consequently, the primary treatment methodologies are administration of adrenaline, infusion of fluids, cardio-pulmonary resuscitation, and time.

In surgery, about 1 in 10,000 patients suffer an anaphylactic reaction to drugs that are administered during the procedure. Compared with typical anaphylactic reactions to allergens to encountered orally or by contact, anaphylactic reactions in surgical environments commonly have a rapid on-set and are severe. Further, when a patient suffers an anaphylactic response to intravenously administered drugs, the histamine released in response to the allergen causes widespread venodilation, which results in a greater blood volume to fill the veins, and less blood volume available to stretch the veins and create venous blood pressure. The drop in venous blood pressure reduces venous return and cardiac output often resulting in anaphylactic shock, which has a reported mortality of 4% despite current management strategies.

There is a need to address the above, and/or at least provide a useful alternative.

### SUMMARY

There is provided a corporeal compression system for use in applying compression to a patient positioned upon a supporting surface, the corporeal compression system comprising:

an overlay having:
flexible sheet material that is arranged into at least two layers to define an internal region therebetween, the layers being repositionable with respect to each other such that the sheet material can assume a deflated state in which the volume of the internal region is a minimum, and
an inlet orifice that opens into the internal region;

one or more restraints that, in use, locate and restrain edge portions of the flexible sheet material relative to the supporting surface with at least part of the patient's body between the supporting surface and the flexible sheet material; and

a gas supply system that has a discharge port that is connected to, or is connectable to, the inlet orifice, the gas supply system being configured to deliver gas through the discharge port,

wherein, in use of the corporeal compression system:

the flexible sheet material of the overlay is draped over the patient so as to provide a posterior layer that is in contact with the patient and an anterior layer that is spaced from the patient by the posterior layer,

the flexible sheet material is restrained relative to the support surface by the restraints, and

the gas supply system is operable to deliver gas via the inlet orifice to increase the volume of the internal region from the deflated state and establish an elevated pressure within the internal region, thereby compressing the patient between the posterior layer of the overlay and the supporting surface.

In at least some embodiments, the gas supply system further comprises one or more of:
a containment vessel that is configured to contain pressurized gas, and a gas distribution circuit interconnects the containment vessel with the discharge port;
a pump having the discharge port through which to discharge gas; and
an inflow connector and gas distribution circuit, the inflow connector interconnecting the discharge port with an independent supply of pressurized gas via the gas distribution circuit,

wherein, in use of the corporeal compression system, the gas supply system is operable to deliver gas to the overlay at a first flow rate and at a second flow rate, wherein the first flow rate is higher than the second flow rate, and wherein the gas supply system is configured to deliver gas at the second flow rate when the pressure within the internal region is above atmospheric.

There is also provided an overlay for use in applying compression to a patient positioned upon a supporting surface, the overlay comprising:

flexible sheet material that is arranged into at least two layers to define an internal region therebetween, the layers being repositionable with respect to each other such that the sheet material can assume a deflated state in which the volume of internal region is a minimum; and

an inlet orifice that opens into the internal region,

wherein, in use of the overlay:

the flexible sheet material in the deflated state is draped over the patient so as to provide a posterior layer that is in contact with the patient and an anterior layer that is spaced from the patient by the posterior layer,

the flexible sheet material is restrained relative to the support surface, and

gas is introduced to the internal region via the inlet orifice to increase the volume of the internal region from the deflated state and establish an elevated pressure within the internal region, thereby compressing the patient between the posterior layer of the overlay and the supporting surface.

Preferably, in use of the corporeal compression system, a portion of the posterior layer conforms to the patient, and the anterior layer is distended when an elevated pressure is established within the internal region.

Preferably, the overlay includes restraints with which to restrain the flexible sheet material to the supporting surface.

In at least some embodiments, the restraints are affixed to the flexible sheet material. In certain embodiments, each restraint is configured to encircle the supporting surface. In such embodiments, each restraints can include a releasable coupling. The releasable coupling can be hook and loop fastener materials. In some alternative embodiments, the releasable coupling is a quick release buckle. Each restraint can include length adjustment.

The layers of the overlay can include an inner layer that is to provide the posterior layer in use of the overlay, and an outer layer that is to provide the anterior layer in use of the overlay. The inlet orifice can be formed in the outer layer. In embodiments in which the restraints are affixed to the flexible sheet material, at least some of the restraints are affixed to the outer layer.

The inner layer can include one or more pleats that extend in the length direction of the overlay. Alternatively or additionally, the inner layer can be made of flexible sheet material that has a higher elasticity in at least one direction than the material of the outer layer. In certain embodiments, the inner layer is made of flexible sheet material that has elasticity in at least a transverse direction of the overlay that is higher than the elasticity of the flexible sheet material of the outer layer. The outer layer can be made of flexible sheet material that includes low elasticity strands. Alternatively or additionally, the overlay can include one or more elongate members that are configured to support hoop stresses in one or more directions from the outer layer when the pressure within the internal region is elevated.

Preferably, the outer layer is made of a flexible sheet material that is substantially inelastic. In certain embodiments, the outer layer is made of a flexible sheet material that includes woven material and a coating that reduces the porosity of the woven material. In some embodiments, the inner layer is made of a flexible sheet material that includes woven material and a coating that reduces the porosity of the woven material. The outer layer can be formed of a material that has a lower gas permeability than the material of the inner layer.

The flexible sheet material can include a superior peripheral edge and an inferior peripheral edge, whereby in use of the overlay the inferior peripheral edge is to be further from the patient's head than the superior peripheral edge. In some embodiments, the overlay is wider at the superior peripheral edge than at the inferior peripheral edge. Alternatively or additionally, the width of the overlay tapers in a direction away from the superior peripheral edge.

Preferably, the overlay has one or more markings to facilitate positioning of the flexible sheet material with respect to the patient at a prescribed position.

The layers of flexible sheet material can be arranged to form distinct peripheral edges to the internal region. In some embodiments, at least some of the peripheral edges of the internal region are spaced internally from the peripheral edges of the outer layer.

The layers of the overlay can be made from separate pieces of flexible sheet material that are joined at the peripheral edges of the internal region.

In some embodiments, the layers of the flexible sheet material are joined at the peripheral edges by at least one of: plastic welding, adhesives, sewn seams. In examples in which the layers of the flexible sheet material are joined at the peripheral edges by sewn seams, the overlay further comprises seal seam material across or within the seams.

In some embodiments, the inlet orifice is part of an inlet connector, and the discharge port is part of an outflow connector that interconnects with the inlet connector. The inlet connector can include an inlet valve that is normally closed. In some embodiments, connecting the outflow connector to the inlet connector causes the inlet valve to open. In some alternative embodiments, the inlet valve includes an actuator that is operable to open the inlet valve.

The overlay can include a plurality of inlet orifices.

In some embodiments, the overlay includes one or more partitions of flexible sheet material that divide the internal region into two or more pockets, wherein the partitions inhibit flow of gas between the pockets, and wherein one of the inlet orifices opening into the internal region within each respective pocket.

In at least some embodiments, the flexible sheet material of the overlay is configured so as to be in the deflated state when draped over the patient.

The overlay can include an overpressure relief valve to vent excess pressure from the internal region to the atmosphere. In some embodiments, the overpressure relief valve opens when the internal pressure within the internal region exceeds a predetermined pressure. In some examples, the predetermined pressure is 60 cm of water or less.

There is also provided a gas supply system for use with an overlay of a corporeal compression system, the flowable material supply system comprising:

a discharge port that is connected to, or is connectable to, the inlet orifice of the overlay,

one or more of:
a pump that is in communication with the discharge port via a conduit;
a containment vessel that is in communication with the discharge port via a gas distribution circuit, the containment vessel being configured to contain pressurized gas; and
a gas distribution circuit that includes an inflow connector that is to interconnect with an independent supply of pressurized gas, and one or more conduits that direct gas from the inflow connector to the discharge port;

wherein in use of the gas supply system within the corporeal compression system:

the gas supply system is operable to deliver gas to the overlay at a first flow rate and at a second flow rate, the first flow rate being higher than the second flow rate, and

the gas supply system is configured to deliver gas at flow rates up to the second flow rate when the pressure within the internal region is above a pre-determined pressure.

In some embodiments in which the gas supply system includes a pump, the pump preferably includes:

an electric motor connected to a rotor that is rotatable to displace gas from an intake, through a chamber in which the rotor is housed, to a discharge port;

a discharge connector that interconnects the discharge port with a complementary connector that is in communication with the inlet orifice of the overlay;

at least one of a flow sensor and a pressure sensor located between the chamber and the discharge port;

a controller that controls the operation of the electric motor, the controller being configured to receive information from the flow sensor and/or the pressure sensor, and to drive the electric motor to vary the flow rate of gas to the discharge port in response to the received information; and

at least one of: a self-contained source of electrical power, and a connector with which to connect the pump to an independent source of electrical power, that provides electrical power to the electric motor.

The controller can be configured such that the pump is operable at a first flow rate to inflate the overlay, and at flow rates up to a second flow rate to establish and/or maintain an elevated pressure within the internal region, the first flow rate being higher than the second flow rate.

In certain embodiments, the controller is configured so that when initialised, the controller is set to initially drive the electric motor to supply gas to the discharge port at the first flow rate.

In some embodiments, the controller is configured so that when initialised, the controller drives the electric motor to supply gas to the discharge port at the first flow rate for a pre-determined period of time. In some embodiments in which the pump includes a flow sensor, the controller is configured so that when initialised, the controller drives the electric motor to supply gas to the discharge port at the first flow rate to discharge a pre-determined volume of gas, and thereafter drives the electric motor to supply gas to the discharge port at flow rates up to the second flow rate. In some in which the pump includes a pressure sensor, the controller is configured so that when the sensed pressure is at or below a pre-determined threshold pressure, the controller drives the electric motor to supply gas to the discharge port at the first flow rate to discharge a pre-determined volume of gas, and when the sensed pressure is above the pre-determined threshold pressure, the controller drives the electric motor to supply gas to the discharge port at flow rates up to the second flow rate.

In at least some embodiments, the controller has a pre-determined set point pressure and the controller is configured to operate to the electric motor to vary the flow rate of gas to the discharge port to maintain the pressure within the internal region of the overlay at the set point pressure. Preferably, the pump has an input user interface that enables a user to set the pre-determined set point pressure. Alternatively or additionally, the set point pressure is adjustable during operation of the pump.

Preferably, the pump has a default set point pressure, and the controller is initialised with the pre-determined set point pressure being the default set point pressure.

In some embodiments, the pre-determined threshold pressure is less than the set point pressure. Alternatively or additionally, the pre-determined threshold pressure is a proportion of the set point pressure.

In some embodiments of the corporeal compression system in which the gas supply system comprises an electrically powered pump:

the pump includes an outflow connector that forms the discharge port, and an electrical switch that is operable to activate the electric motor of the pump; and

the overlay includes a conduit that is interconnected at a first end to the flexible sheet material so as to open into the inlet orifice, the second end of the conduit including an inlet connector that is releasably couplable to the outflow connector,

wherein the action of coupling the inlet connector with the outflow connector operates the electrical switch.

Preferably, the inlet connector and outflow connector are configured such that an elevated pressure within the conduit biases the inlet connector and outflow connector into the coupled state. Alternatively or additionally, the pump can include a spring that is positioned to bias the inlet connector and outflow connector into the coupled state.

In at least one form, the inlet connector and outflow connector form a bayonet mount, and the electrical switch is positioned relative to the outflow connector so as to be actuated after the first movement of coupling the inlet connector and outflow connector is complete. Alternatively or additionally, the electrical switch is positioned relative to the outflow connector so as to be actuated during the first movement of decoupling the inlet connector and outflow connector.

In some embodiments in which the gas supply system includes a containment vessel, the gas supply system further includes:

a discharge flow regulator that regulates the flow of gas from the containment vessel to the discharge port, the regulator being configured:
to discharge gas to the discharge port at flow rates up to the first flow rate when the pressure within the internal region is at or below a threshold pressure, and
to discharge gas to the discharge port at flow rates up to the second flow rate when the pressure within the internal region is above the threshold pressure and below the pre-determined pressure and/or the selected set point pressure.

In some embodiments, the first flow rate corresponds with substantially unregulated discharge of gas from the containment vessel to the discharge port.

In some embodiments, the discharge flow regulator has a plurality of pneumatically operated valves that are in fluid communication with the internal region via the inlet orifice and the discharge port, wherein each valve is operable to be open at a unique threshold pressure. The discharge flow regulator can include a first stage regulator that when open regulates flow of gas out of the containment vessel at flow rates up to the first flow rate, and a second stage regulator that regulates flow of gas to the discharge port to flow rates up to the second flow rate when the pressure within the internal region is above the pre-determined pressure, wherein the pre-determined pressure is less than a set point pressure.

In some alternative embodiments, the discharge flow regulator includes:

one or more electrically operated valves;

at least one of a flow sensor and a pressure sensor located between the chamber and the discharge port;

an electronic controller that controls the operation of the valves, the controller being configured to receive information from the flow sensor and/or the pressure sensor, and operate valves in response to the received information; and

at least one of: a self-contained source of electrical power, and an independent source of electrical power, that provides electrical power to the electronic controller.

There is also provided an overlay for use in applying compression to a patient positioned upon a supporting surface, the overlay comprising:

flexible sheet material that is arranged into at least two layers to define an internal region therebetween, the layers being repositionable with respect to each other such that the sheet material can assume a deflated state in which the volume of internal region is a minimum;

a compressible open cell material contained within the internal region;

an inlet orifice that opens into the internal region;

an inlet valve that is operable to selectively allow passage of air through the inlet orifice; and

restraints with which to restrain the flexible sheet material to the supporting surface, the restraints being length adjustable,

wherein, in use of the overlay:

the inlet valve is opened to allow the compressible open cell material to fill the internal region, and then closed to isolate the internal region from the atmosphere;

the overlay is placed over the patient so as to provide a posterior layer that is in contact with the patient and an anterior layer that is spaced from the patient by the posterior layer, and

the restraints are used to restrain the flexible sheet material relative to the support surface, and the length of the restraints are adjusted to establish tensile forces in the anterior layer so as to establish an elevated pressure within the internal region, thereby compressing the patient between the posterior layer of the overlay and the supporting surface.

In such embodiments, the overlay includes at least one overpressure relief valve to vent excess pressure from the internal region to the atmosphere.

### BRIEF DESCRIPTION OF THE DRAWINGS

In order that the invention may be more easily understood, embodiments will now be described, by way of example only, with reference to the accompanying drawings, in which:

FIG. 1: is a schematic view of a corporeal compression system according to a first embodiment of the present invention;

FIG. 2: is a plan view of the anterior side of the overlay of the corporeal compression system of FIG. 1;

FIG. 3: is a plan view of the posterior side of the overlay of FIG. 2;

FIG. 4: is a schematic cross section view of the corporeal compression system as viewed along the line A-A in FIG. 1;

FIG. 5: is a schematic cross section view of the corporeal compression system as viewed along the line B-B in FIG. 1;

FIG. 6: is a schematic cross section view of the corporeal compression system as viewed along the line A-A in FIG. 1, showing the overlay in a deflated state;

FIG. 7: is a schematic block diagram of the pump of the corporeal compression system of FIG. 1;

FIG. 8: is a view of the User Interface of the pump of the corporeal compression system of FIG. 1;

FIG. 9: is a chart showing overlay fill volume and internal region pressure against time for a corporeal compression system of FIG. 1;

FIG. 10: is a schematic view of a gas supply system according to a second embodiment of the present invention;

FIG. 11: is a schematic view of a gas supply system according to a third embodiment of the present invention;

FIG. 12: is a schematic view of a corporeal compression system according to a fourth embodiment of the present invention;

FIG. 13: is a plan view of the anterior side of an overlay fir a corporeal compression system according to a fifth embodiment of the present invention; and

FIG. 14: is a plan view of the posterior side of the overlay of FIG. 13.

### DETAILED DESCRIPTION

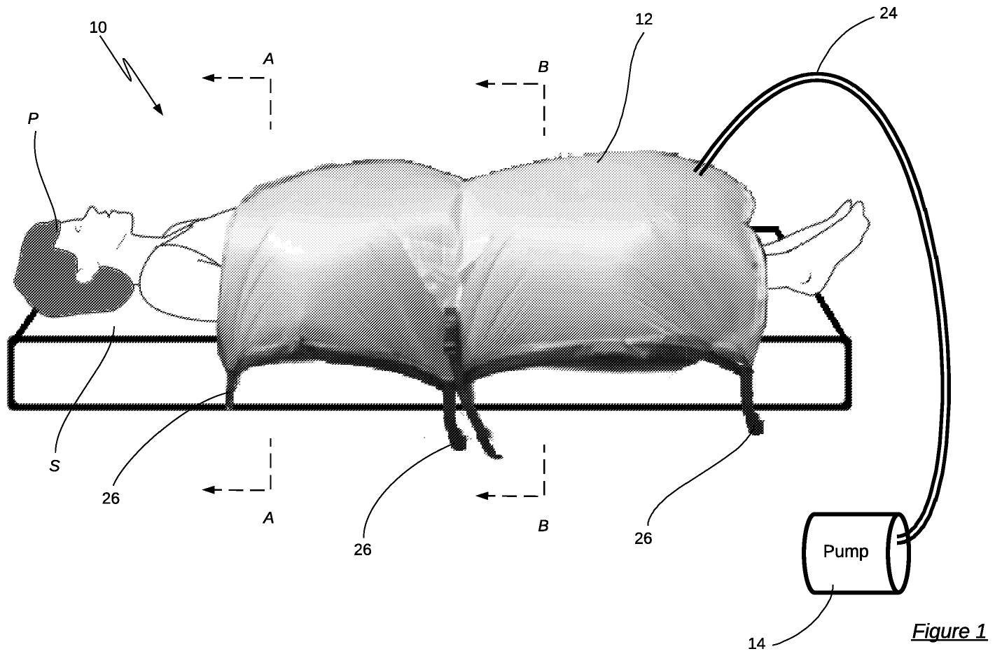

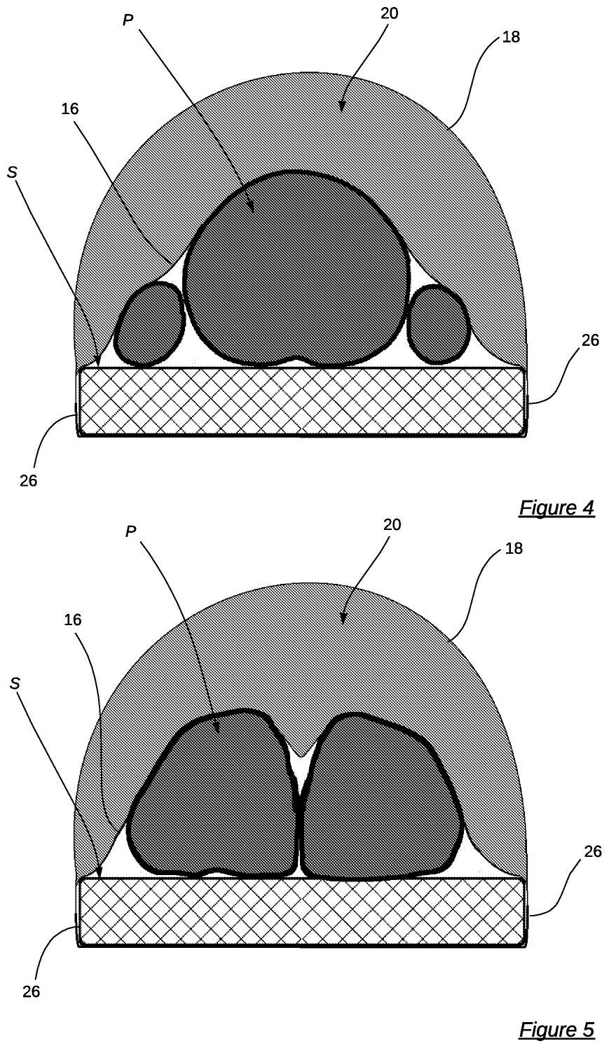

FIGS. 1 to 6 show a corporeal compression system 10 according to an embodiment of the present invention. The compression system 10 in use is to apply compression to a patient P positioned upon a supporting surface S, which may be for example the upper surface of a theatre bed.

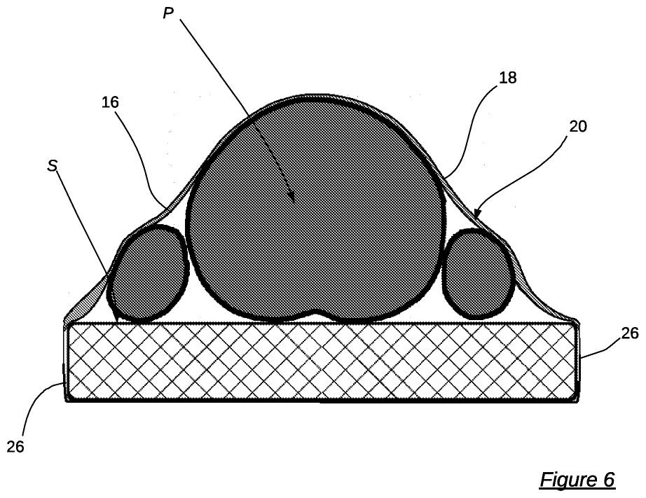

The compression system 10 has an overlay 12, and a flowable material supply system, which in this embodiment is in the form of a pump 14. The overlay 12 has flexible sheet material that is arranged into layers 16, 18. An internal region 20 is defined between the layers 16, 18. As the sheet material of the layers 16, 18 is flexible, the layers can be repositioned with respect to each other. In this way, the sheet material of the overlay 12 can assume a deflated state in which the volume of the internal region 20 is a minimum. FIG. 6 shows schematically the overlay 12 in a deflated state, and draped over the patient P. In this particular embodiment, the flexible sheet material of the overlay 12 is arranged to form a posterior layer 16 that is in contact with the patient P when the overlay 12 is draped over the patient P, and an anterior layer 18 that is spaced from the patient P by the posterior layer 16.

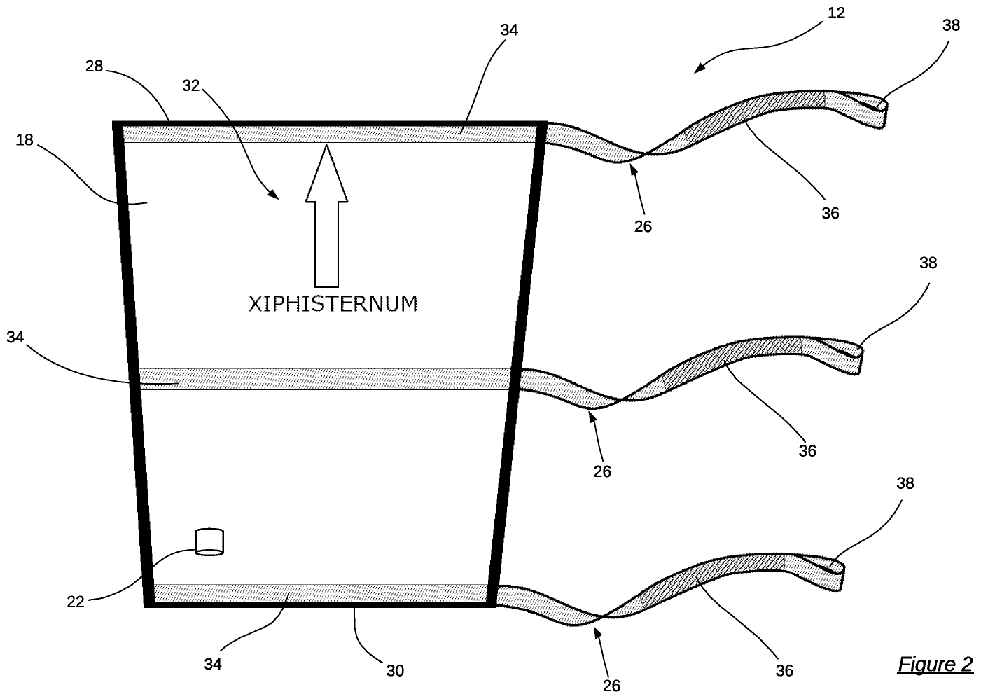

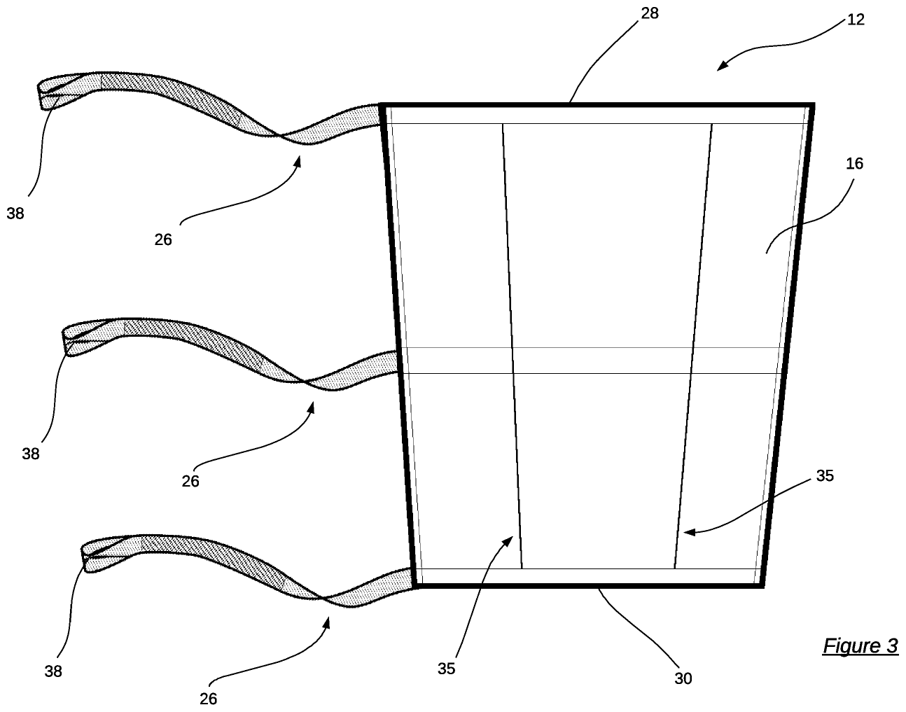

FIG. 2 shows the anterior side of the overlay 12, and thus the anterior layer 18. FIG. 3 shows the posterior side of the overlay 12, and thus the posterior layer 16.

As shown in FIG. 2, the overlay 12 further has an inlet connector 22 within the anterior layer 18. The inlet connector 22 defines an inlet orifice that opens into the internal region 20. The pump 14 has an outlet pipe 24 that, in this particular embodiment, is releasably connectable to the inlet connector 22. In this way, gas from the pump 14 is delivered through the outlet pipe 24, and into the internal region 20.

In this example, the pump 14 has an intake (not shown) that draws in atmospheric air, and a rotor (not shown) that is rotatable to displace gas from the intake, through a chamber (not shown) in which the rotor is housed, to the discharge port.

The compression system includes restraints 26 that are to locate and restrain lateral edge portions of the flexible sheet material of the overlay 12 relative to the supporting surface S. In this particular embodiment, the restraints 26 are integral with overlay 12. In FIGS. 1 and 4 to 6, the restraints 26 extend underneath, and thus around, the theatre bed.

In use of the corporeal compression system 10, the overlay 12 in the deflated state is draped over the patient P, such that the posterior layer 16 is in contact with the patient P, and also with the support surface S. The anterior layer 18 faces outwardly and away from the patient P. The overlay 12 is restrained relative to the support surface S by the restraints 26. The pump 14 is then operated to deliver air via the inlet orifice to increase the volume of the internal region 20 from the deflated state. Once the internal region 20 has been filled to the available capacity, the pump 14 then establishes an elevated pressure within the internal region 20. The pressure differential between the internal region 20 and the surrounding atmosphere, together with tensile forces generated in the restraints 26, compress the patient P between the posterior layer 16 of the overlay 12 and the supporting surface S. Due to the flexibility of the sheet material, the posterior layer 16 conforms at least in part to the patient's body. In this regard, it will be appreciated that, due to various factors, there will be air gaps in certain regions between the patient P and the overlay, and in some instances also between the supporting surface S. Notwithstanding such air gaps, the compressive forces applied by the overlay 12 are distributed across the substantially around the external surfaces of the patient's body that are facing away from the supporting surface S.

As shown in FIG. 1, the superior edge 28 of the overlay 12 is to be positioned approximately level with the patient's xiphisternum, when the overlay 12 is in its deflated state and draped over the patient P. The inferior edge 30 of the overlay 12 (which is the edge furthest from the patient's head) will locate in a position on the patient that is determined by the height of the patient P and the length of the overlay 12. To facilitate the correct optimal location of the overlay 12 on a patient, the anterior layer 18 of the overlay 12 has markings 32 to facilitate positioning of the flexible sheet material with respect to the patient at a prescribed position. As shown in FIG. 2, in this example, the markings 32 consist of the word “XIPHISTERNUM” and an arrow with the tip pointing to the centre of the superior edge 28. In the illustrated example, the inferior edge 30 of the overlay 12 is positioned proximally of the patient's ankles.

As will be apparent, when the overlay 12 is inflated and pressurized, the compression system 10 applies pressure that compresses the patient P and effectively “squeezes” the portion of the patient body that is beneath the flexible sheet material. Depending on the magnitude of the pressure applied by the compression system 10, there are a variety of advantages that may be obtained through use of the compression system 10. For a patient experiencing widespread venodilation, compression in this manner can increase the patient's venous return, which has the consequence of increasing cardiac output. Preliminary trials indicate that compression achieved by air pressures within the internal region 20 of up to, and including 60 centimetres of water (hereinafter “cm H₂O”) is beneficial in redistributing venous blood to restore functional cardiac output where venodilation is present. In this regard, the preliminary trials indicate that compression achieved by air pressures within the internal region 20 in the range of 15 to 45 centimetres of water (hereinafter “cm H₂O”) is particularly beneficial. Further, the trials suggest that compression achieved by air pressures within the internal region 20 in the range of 25 to 35 centimetres of water (hereinafter “cm H₂O”) may be highly effective in treatment of venodilation.

In the illustrated example, the squeezed portion of the patient P is their abdomen and legs. A large proportion of a person's venous blood volume is stored in a person's abdomen and legs. Use of the compression system 10 on a patient as illustrated in FIG. 1 can redistribute a patient's venous blood to their head and chest region.

By way of example, a patient suffering an anaphylactic reaction during a surgical procedure to anaesthetic drugs that have been administered intravenously is likely to have had the allergen delivered rapidly through their blood stream. The widespread presence of the allergen can induce a histamine response through much of their body. The subsequent venodilation will rapidly decrease venous blood pressure, which then lowers venous return and thus limits cardiac output. In severe cases, extensive venodilation can result in the patient's death. Use of the compression system 10 facilitates the management of the anaphylactic reaction in this setting by redistributing the venous blood, which may avoid cardiac arrest due to loss of cardiac output. With the patient stabilized by the external compression, the patient's natural histamine response, possibly augmented with an infusion of adrenaline, has sufficient time to reverse the anaphylactic response. In other words, the corporeal compression system can provide additional care to the use of adrenaline, intravenous fluids and time, in the treatment of an anaphylactic response.

As will be appreciated, the compression system 10 of this embodiment utilizes the supporting surface Son which the patient P is lying in the application of compression. This has the distinct advantage of minimizing, if not eliminating, need to move the patient in the fitting of the overlay to the patient.

With regard to a patient who has sustained certain injuries, such as internal venous bleed due to a pelvic fracture, the application of compression using the compression system 10 of this embodiment can limit venous bleeding in the abdomen and/or lower limbs. As will be apparent, limiting venous bleeding can improve the prospects of recovery from the injuries. By way of example, pelvic fractures are frequently accompanied by internal bleeding, which in some cases occurs from the venous circulation. As pelvic fractures are usually the result of accidental trauma, attending emergency medical services (EMS) team initially stabilize the patient at the accident location, prior to transporting the patient to a hospital. While transporting the patient, it may be necessary to introduce fluids to compensate for the venous bleeding and maintain venous return. A corporeal compression system according to an embodiment of the invention can be used by the EMS team during the transport, to limit venous bleeding and then limit the fluid infusion required. To this end, the patient can be loaded onto the EMS stretcher, and the overlay of the compression system draped over the patient and restrained to the EMS stretcher using the system's restraints. Once the flexible sheet material is inflated and pressurized, the overlay together with the stretcher co-operate to compress the patient, the compression operating to limit the extent of venous bleeding.

It will be appreciated that the compression system of embodiments can alternatively or additionally be used in transporting patients with other injuries. One particular benefit being that the stability of the patient with respect to the support surface on which they are positioned can be enhanced by the compression, which can assist in limiting the patient's discomfort.

A further benefit of use of the compression system in transport is that it operates in a manner similar to the safety restraints that are often fitted to a patient during transport.

To facilitate movement of the posterior layer 18 during inflation, the posterior layer 18 is provided with a pair of pleats 35. Each pleat 35 extends in the length direction of the overlay 12.

As will be appreciated, the available capacity (in other words, the maximum available volume) of the internal region depends on several factors, including the size of the patient P (and in particular their girth), the geometries of the supporting surface S and the anterior layer 16, and the elasticity of the anterior layer 16. The anterior layer 16 in some embodiments may be made of a flexible sheet material that is substantially inelastic. This has the benefits of minimizing stretching of the anterior layer, which increases the available capacity, avoiding a change in permeability of the anterior layer (which can occur with some materials when stretched), and/or minimizing the likelihood of the anterior layer material tearing.

In one example, the anterior layer 16 is made of a flexible sheet material that includes woven material and a coating that reduces the porosity of the woven material. The coating may be, for example, a polymer coating, such as a polyurethane or acrylics materials, may be applied to the woven material during manufacture of the sheet material. Such polymer coatings can be beneficial in blocking pores in the sheet material, and thus limiting gas permeability. Similarly, with regard to the posterior layer 18.

In this particular embodiment, the overlay 12 is wider at the superior peripheral edge 28 than at the inferior peripheral edge 30. Further, the width of the overlay 12 tapers in a direction away from the superior peripheral edge 28. This has the benefit of maximizing the contact surface between the overlay 12 and the patient P, while also minimizing the maximum volume of the internal region 20.

In the illustrated example, layers 16, 18 of the overlay 12 are made from separate pieces of flexible sheet material that are joined at the peripheral edges of the internal region 20. In this way, the layers 16, 18 of flexible sheet material form distinct peripheral edges to the internal region 20.

The restraints 26 are affixed to the flexible sheet material of the overlay 12. In the embodiment as illustrated in FIGS. 2 and 3, each restraint 26 has a length that is greater than that required to encircle the supporting surface S when a patient P is resting on that the surface. Each restraint 26 has a releasable coupling that, in this embodiment is in the form of hook and loop fastener materials 34, 36. In this example, the hook material 34 is provided on a portion of the “free” section of the restraint 26. The loop material 36 is provided across the width of the flexible sheet material, and on the external surface of the anterior layer 18.

To facilitate restraining the overlay 12 to a bed that provides the support surface 5, there is a looped handle 38 at the terminal end of each restraint 26. When locating and restraining the overlay 12, the “free” section of each restraint 26 is passed underneath the support surface 5, and then positioned to interconnect the hook and loop fastener materials 34, 36. Two surgical attendants can quickly pass the looped handle 38 beneath the patient P and support surface S while fitting the overlay 12.

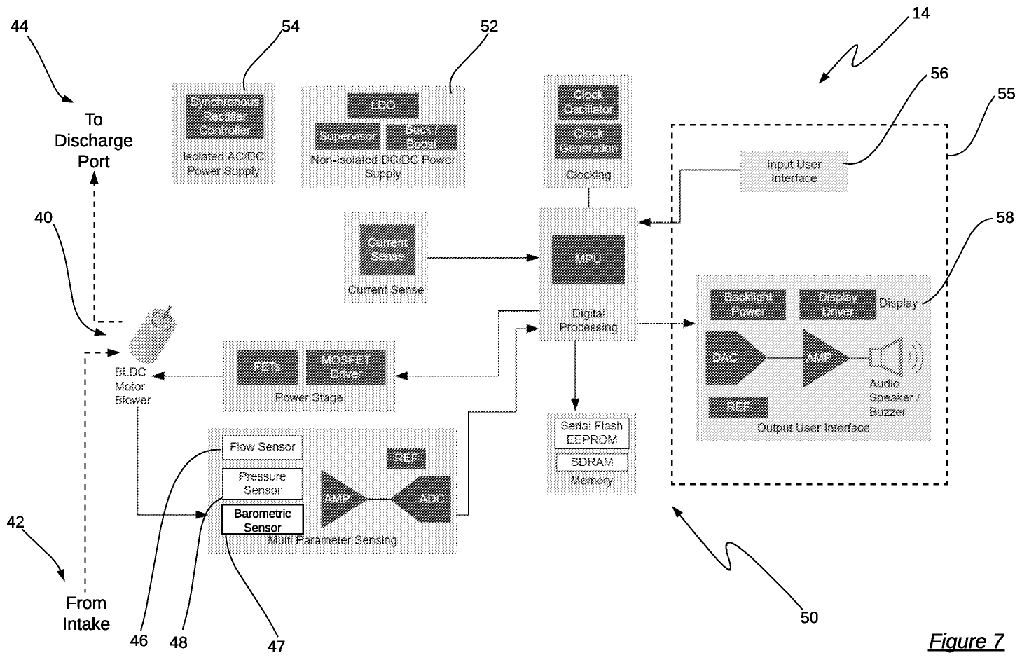

FIG. 7 is a block diagram of the components of the pump 14 of the corporeal compression system 10 of FIG. 1.

The pump 14 of this embodiment includes a brushless DC Motor Blower 40. Within the motor blower 40 is an electric motor that is connected to a rotor that is rotatable within a chamber. The Motor Blower 40 displace air that is drawn from an intake 42, through the chamber by the rotation of the rotor, and discharged to a discharge port 44. As previously described, the pump 14 includes an outlet pipe 24, and in this particular embodiment, the discharge port 44 is formed at the inner terminal end of the outlet pipe 24 that is permanently connected to the pump housing. At the outer end of the outlet pipe 24, the pump 14 has discharge connector (not shown) that interconnects the discharge port with the (complementary) inlet connector 22 that is in communication with the inlet orifice of the overlay 12.

The pump 14 includes a flow sensor 46 and a pressure sensor 48 that are located between the chamber and the discharge port of the pump 14. In this way, the flow sensor 46 measures the flow rate of air discharged from the pump 14. From information obtained from the flow sensor 46, the fill volume of the internal region 20 of the overlay 12 can be at determined, at least with sufficient accuracy. The pressure sensor 48 measures the pressure of air discharged from the pump 14. As will be appreciated, the air pressure is substantially similar to the internal pressure within the internal region 20.

The pump 14 further has a controller 50 that controls the operation of the Motor Blower 40. As indicated in FIG. 7, the controller 50 is configured to receive information from the flow sensor 46 and the pressure sensor 48. Accordingly, the controller 50 is able to vary the electric motor speed to vary the flow rate of air to the discharge port, in response to the received information.

In this particular embodiment, the pump 14 also a self-contained source of electrical power, which in this embodiment is a non-isolated power supply 52 such as a battery. An electrical connector (not shown) is also provided, with which to connect the pump to an independent source of electrical power, such as a mains AC power supply. The electrical connector is coupled to an isolated AC/DC power supply 54. In this way, the pump 14 can be powered by either a battery, or from mains AC power supply.

The controller 50 of this embodiment is configured such that the pump 14 is operable at a first flow rate to inflate the overlay 12, and at flow rates up to a second flow rate to establish and/or maintain an elevated pressure within the internal region 20. The first flow rate is higher than the second flow rate. In particular, the first flow rate can be utilized to provide a high-volume flow into the internal region 20, which can enable rapid inflation of the overlay 12. Once the maximum available capacity of the internal region 20 has been reached, the pump 14 can then be operated at the second flow rate that establishes and/or maintains the elevated pressure within the internal region 20. As will be appreciated, the second flow rate can ideally match the air leakage from the overlay 12, and in embodiments in which the air leakage is negligible, the pump 14 may be operable from flow rates that approach nil.

Once the maximum available capacity of the internal region 20 has been reached, the controller 50 can operate in a cyclical manner, involving alternating between the second flow rate and no output, in order to maintain the elevated pressure. Alternatively, the controller 50 can utilize a feedback loop control system in which the second flow rate is adjusted based on inputs from the pressure sensor 48, and in some cases also the flow sensor 46.

The controller 50 can also be configured so that, when initialised, the Motor Blower 40 is initially driven to supply air to the discharge port at the first flow rate. In this way, the controller 50 is operating on the initialisation assumption that the overlay 12 is in its deflated state. A barometric pressure sensor 47 is also be provided, which enables comparison of the atmospheric pressure (obtained via the barometric pressure sensor) and data obtained by the pressure sensor 48. In particular, the pressure differential between atmospheric pressure, and the pressure at the outlet of the Motor Blower 40.

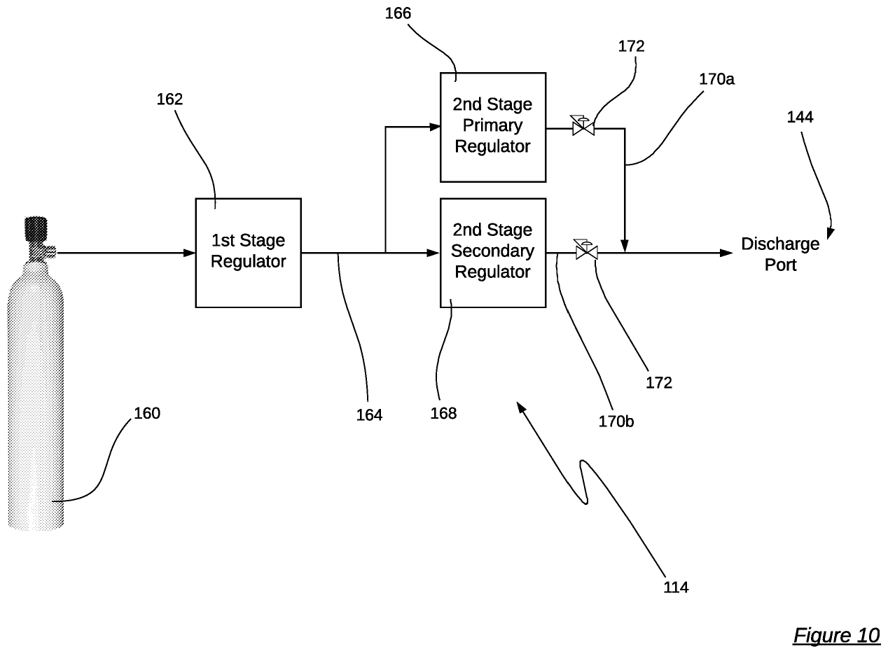

FIG. 9 is a chart showing the overlay 12 fill volume (shown in a plotted solid line of the chart, and indicated by arrow V) and internal region pressure (shown in a plotted dash line of the chart, and indicated by arrow D), against time on the horizontal axis. FIG. 9 also shows the flow rate of air discharged by the pump 14 (shown in a plotted dash-dot line of the chart, and indicated by arrow F).

At time T=0, the overlay 12 is in its deflated state, and thus the fill volume V and internal region pressure D are both zero. Time T=0 represents the time at which the gas supply system is activated. In this example, the gas supply system delivers a substantially constant high flow rate of air to the overlay 12 between time T=0 and time T=t1. Accordingly, during this period the overlay 12 is being inflated at a substantially constant rate (which is the higher first flow rate of the pump 14) and the fill volume V increases substantially linearly from zero to a volume approaching the maximum available capacity.

At time T=t1, the overlay 12 is approaching its available capacity, and hence between time T=t1 and time T=t2 the internal region pressure D increases, in a non-linear manner, from zero to an elevated pressure. Accordingly, during this period in which the internal region 20 of the overlay 12 is being pressurized to the elevated pressure, and the pump 14 is operating at the lower second flow rate.

After time T=t2, the internal region pressure D is to be maintained at the elevated pressure. As will be appreciated, air leakage from the overlay 12 will cause the pressure within the internal region 20 to fall over time in the absence of an inflow of air into the internal region 20. Accordingly, after time T=t2, the pump 14 is operated at flow rates that are up to the second flow rate.

In this example, the controller 50 is configured so that when the sensed pressure—obtained from the pressure sensor 48—is above a pre-determined threshold pressure, the controller 50 drives the Motor Blower 40 to switch the flow rate of discharged air from the first flow rate, to flow rates up to the second flow rate. The pre-determined threshold pressure in this example is sensed at time T=t1.

It will be appreciated that the chart of FIG. 9 is schematic only, and illustrative of one manner in which the pump 14 may be operated.

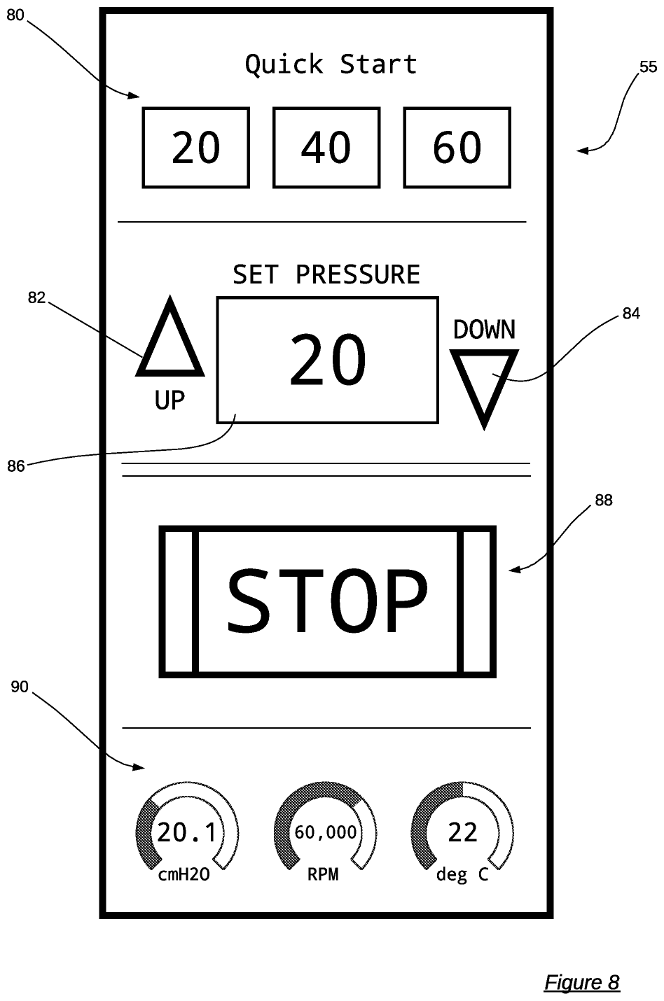

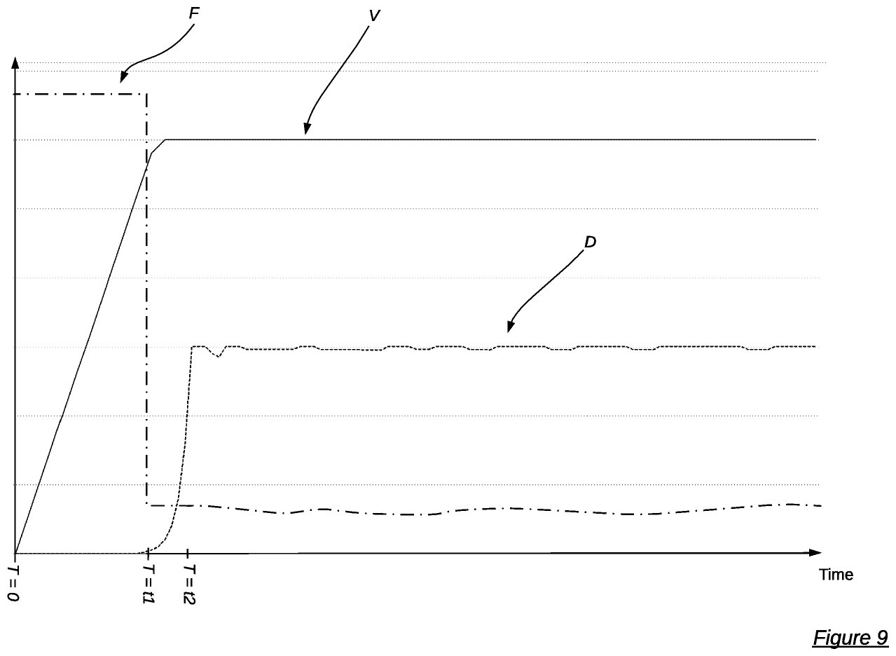

As shown in FIG. 7, the pump 14 has a user interface 55, that includes an input user interface 56 that enables a user to operate the pump, and an output user interface 58 that provides visual information for the user to ascertain the operational status of the pump 14. In one example, the user interface 55 includes a touchscreen display, as illustrated in FIG. 8.

As shown in FIG. 8, the user interface 55 enables the user to set a pre-determined set point pressure, which is the desired maximum elevated pressure of the internal region 20. The input user interface 58 can enable a user to adjust the set point pressure during operation of the pump 14. To this end, the user interface 55 has “Quick Start” inputs 80 that allow selection of operation of the pump to initial set point pressures of 20 cm H₂O, 40 cm H₂O, and 60 cm H₂O. The set point pressure can be adjusted, either decreasing the set point pressure using a pressure decrease input 82, or increasing the set point pressure using a pressure decrease input 84. A user can immediately cease operation of the Motor Blower 40 via a “STOP” input 88.

The output user interface 58 portion of the user interface 55 shows the “SET PRESSURE” in display region 86. Further, the output user interface 58 portion of the user interface 55 includes a pump operating parameter display portion 90 that includes digital gauges and numerical values for each of the sensed pressure (via the pressure sensor 48), the Motor Blower 40 rotational speed, and the temperature of air flowing through the Motor Blower 40.

In this particular embodiment, the output user interface 58 also provides information audibly via a speaker from which the user can ascertain the operational status of the pump 14.

As will be appreciated, compression systems of embodiments of the invention can be used in the treatment of other conditions, including (but not limited to) distributive shock, hypotension, and external venous bleeding in the abdomen and/or lower limbs. It will be appreciated that the level of compression, which is correlated with the internal pressure of the internal region, may be different depending on many factors, including (but not limited to) the condition being treated, the instant event being treated, particulars of the individual being treated.

Furthermore, it is known that compression is beneficial in managing lactic acid build up in soft tissues. A compression system according to certain embodiments may be efficacious in sports recovery. In such embodiments, it may be desirable for the air that is delivered to the internal region to be chilled in order to provide the dual benefits of compression and cold therapy. In such circumstances the compression system may include a heat exchanger that is configured to lower the temperature of air that is being delivered to the internal region. Such embodiments may include an air return line from the overlay to the intake to the pump. The gas supply system may optionally include an atmospheric air intake and a valve that switches the intake air being drawn into the pump from atmospheric air and air from the air return line. The heat exchanger can be provided on any of the air return line, the intake to the pump downstream of the valve, and the air discharge line from the pump to the overlay. In this way, air within the internal region of the overlay may be kept at a temperature below ambient temperature.

FIG. 10 shows schematically a gas supply system 114 according to a further embodiment. The gas supply system 114 includes a containment vessel, which in this embodiment is in the form of a gas cylinder 160, and a discharge flow regulator. The discharge flow regulator is configured to regulate the flow of gas from the gas cylinder 160 to the discharge port 144. The gas supply system 114 also includes conduits, such as hoses (not shown in FIG. 10), that interconnect various components of the gas supply system 114. The gas supply system 114 also includes a discharge connector (not shown) that interconnects the discharge port 144 with the (complementary) inlet connector that is in communication with the inlet orifice of the overlay.

The regulator is configured to discharge gas to the discharge port 144:
a. at flow rates up to a first flow rate, when the pressure within the internal region is within a first pressure range that includes atmospheric pressure and up to a threshold pressure, and
b. at flow rates up to a second flow rate, which is lower than the first flow rate, when the pressure within the internal region is within a second pressure range that is above the threshold pressure and a pre-determined pressure and/or the selected set point pressure.

The pre-determined pressure/selected set point pressure is greater than the threshold pressure; and the threshold pressure is greater than atmospheric pressure.

In the illustrated embodiment, the regulator includes a 1^{st} Stage Regulator 162 that reduces the pressure of gas from the gas cylinder 160. As indicated schematically in FIG. 10, a conduit 164 on the outlet side of the 1^{st} Stage Regulator 162 branches to a 2^{nd} Stage Primary Regulator 166, and a 2^{nd} Stage Secondary Regulator 168. On the discharge port side of each of the 2^{nd} Stage Primary and Secondary Regulators 166, 168, are a pair of conduits 170 that join to lead to the discharge port 144.

As will be appreciated, in use of the gas supply system 160 within a corporeal compression system, the pressure within the pair of conduits 170 is substantially equal to the internal pressure of the internal region of the overlay. The 2^{nd} Stage Primary Regulator 166 is configured to gas at a high flow rate (which is the first flow rate) into conduit 170a, and so to the discharge port 144. The 2^{nd} Stage Primary Regulator 166 is a demand valve that is open when the pressure within the conduit 170a is below the threshold pressure. When the pressure within the conduit 170a rises above the threshold pressure, the 2^{nd} Stage Primary Regulator 166 closes.

The 2^{nd} Stage Secondary Regulator 168 is configured to gas at a low flow rate (which is the second flow rate) into conduit 170b, and so to the discharge port 144. The 2^{nd} Stage Secondary Regulator 168 is a demand valve that is closed when the pressure within the conduit 170b is at atmospheric pressure. As the pressure within the conduit 170b rises and approaches the threshold pressure, the 2^{nd} Stage Secondary Regulator 168 opens. Further, when the pressure within the conduit 170b is at the pre-determined pressure/selected set point pressure, the 2^{nd} Stage Secondary Regulator 168 closes, such that there is no gas flowing to the discharge port 144. As will be appreciated, it is advantageous for both the 2^{nd} Stage Primary and Secondary Regulators 166, 168 to be open in a narrow pressure range that includes the threshold pressure, to ensure continuity of flow to the overlay during inflation.

The gas supply system 160 includes a pair of valves 172 on the pair of conduits 170, which provides the capacity to manually adjust the flow rate through either conduit 170a, 170b if desired.

The 2^{nd} Stage Secondary Regulator 168 can include an adjuster that enables the set point pressure to be adjusted, if desired.

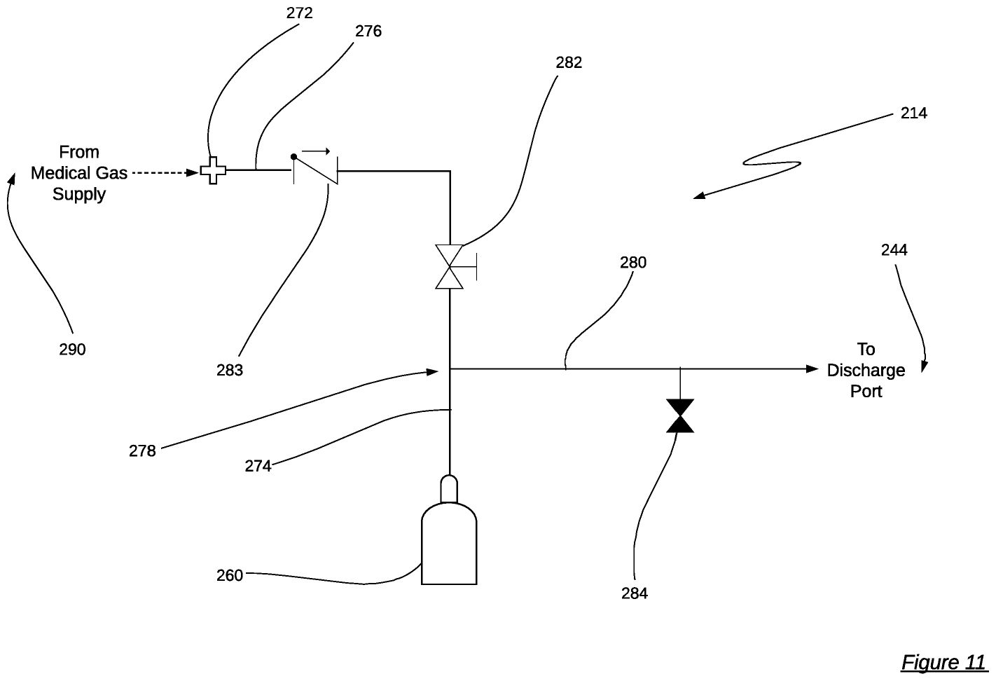

FIG. 11 shows schematically a gas supply system 214 according to another embodiment. The gas supply system 214 includes a contained source of pressurized gas, which in this embodiment is in the form of a gas cylinder 260, and a gas distribution circuit 270 that includes an inflow connector 272 that is to interconnect with an independent supply of pressurized gas, such as a medical gas supply line 290.

The gas supply system 214 includes a first conduit 274 that is connected to the outlet of the gas cylinder 260, a second conduit 276 that is connected to the inflow connector 272. The first and second conduits 274, 276 are joined at a junction 278, and a third conduit 280 extends from the junction 278 to the discharge port 244. In this way, gas can flow from the medical gas supply line 290, through the inflow connector and the second and third conduits 276, 280, to the discharge port 244. Further, gas can also flow from the gas cylinder 260, through the first and third conduits 274, 280, to the discharge port 244.

A gate valve 282, and a check valve 283 are provided in the second conduit 276. In this example, the gas cylinder 260 is a single use cylinder. The action of opening the gate valve 282 simultaneously pierces the seal on the gas cylinder 260 to release gas from within the cylinder. In this embodiment, the flow of gas from the gas cylinder 260 is substantially unregulated. The volume of gas that is contained within the cylinder (at the elevated pressure) is substantially equal to the maximum available capacity of the internal region of the overlay. In this way, the overlay is inflated as gas from the gas cylinder 260 is depleted. Thus, in this particular embodiment, the first flow rate corresponds with the substantially unregulated discharge of gas from the gas cylinder 260.

Once the gas cylinder is substantially depleted, the inflow of gas from the medical gas supply line 290 will dominate, and provide a flow of gas at the lower, second flow rate to the discharge port 244. The gate valve 282 is also configured to operate as a demand valve, whereby flow through the second conduit 276 is only enabled when the pressure within the second conduit 276 is below the set point pressure. The check valve 283 prevents back flow of gas from the system 214 into the medical gas supply line 290.

As will be appreciated, the medical gas supply line 290 may have a suitable pressure to achieve the desired set point pressure in use of the corporeal compression system, but may have a flow rate that is so low that the time to inflate the overlay is unacceptably long. The “hybrid” gas supply that is provided by the gas supply system 214 enables rapid inflation of the overlay using gas from the gas cylinder 260, and also a reliable and continuous supply of pressurized gas from the medical gas supply line 290.

In this particular embodiment, the third conduit 280 includes a bleed valve 284 that releases gas when the pressure within the third conduit 280 exceeds a limit pressure. In the event that the corporeal compression system that includes the gas supply system 214 is used with a patient of particularly large girth, the available capacity may be less than the volume of gas that is contained within the gas cylinder 260 (at the elevated pressure). In this scenario, excess high-pressure gas can be exhausted from the bleed valve 284, which minimizes the likelihood of the overlay attaining an excessive pressure, or damage to the corporeal compression system.

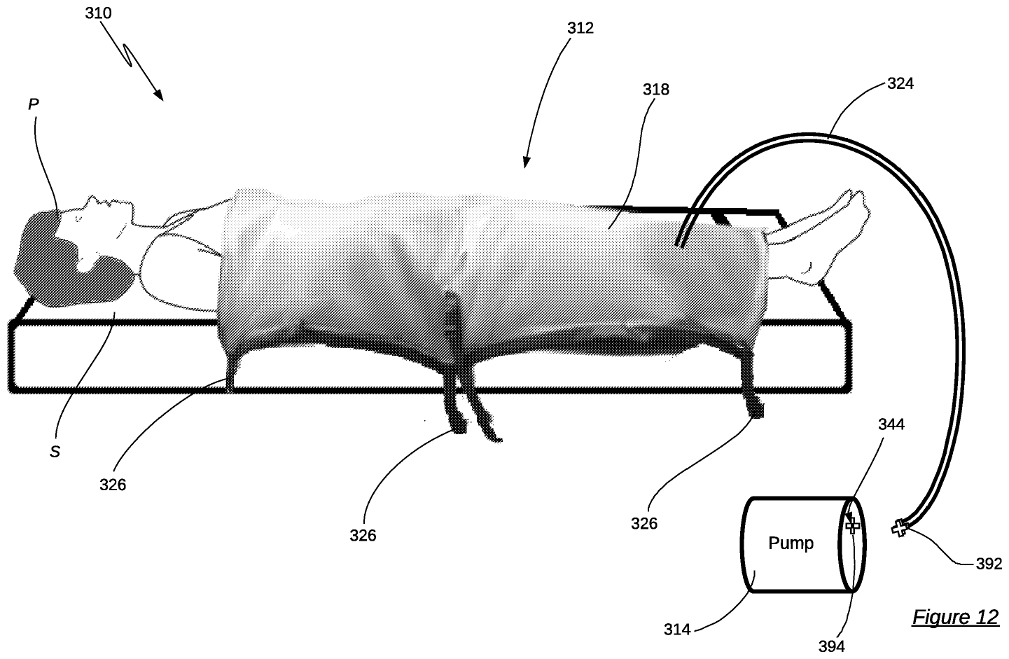

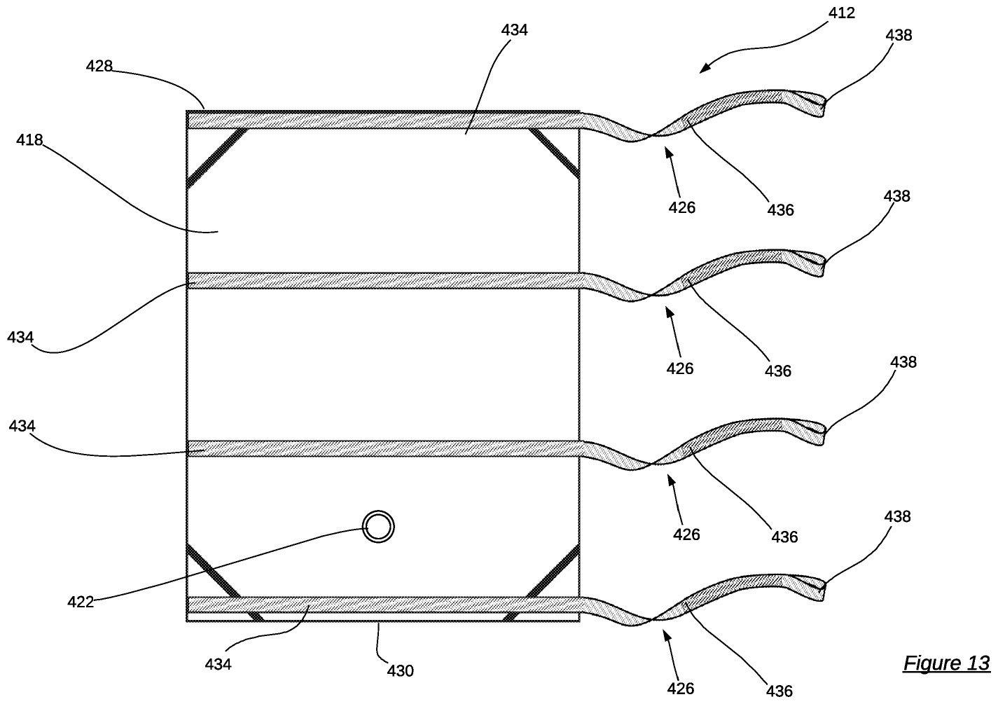

FIG. 12 shows a corporeal compression system 310 according to another embodiment of the present invention. The corporeal compression system 310 is substantially similar to the corporeal compression system 10 of FIG. 1. Accordingly, components of the corporeal compression system 310 that are similar to components of the corporeal compression system 10 have the same number with the prefix “3”.

FIG. 12 shows the overlay 312 in its deflated state. The overlay includes a conduit 324 that is interconnected at its first end to the posterior layer 318 of the flexible sheet material so as to open into the inlet orifice. At the second end of the conduit 324 is an inlet connector 392.

The pump 314 includes an outflow connector 394 that forms the discharge port 344, and an electrical switch (not shown) that is operable to activate the electric motor of the pump 314. The inlet connector 392 is releasably couplable to the outflow connector 394. In this example, the inlet connector 392 and outflow connector 394 form a bayonet mount. The action of coupling the inlet connector 392 with the outflow connector 394 operates the electrical switch.

In this example, the electrical switch is positioned relative to the outflow connector 394 so as to be actuated after the first movement of coupling the inlet connector 392 and outflow connector 394 is complete. Further, the electrical switch is positioned relative to the outflow connector 394 so as to be actuated during the first movement of decoupling the inlet connector 392 and outflow connector 394.

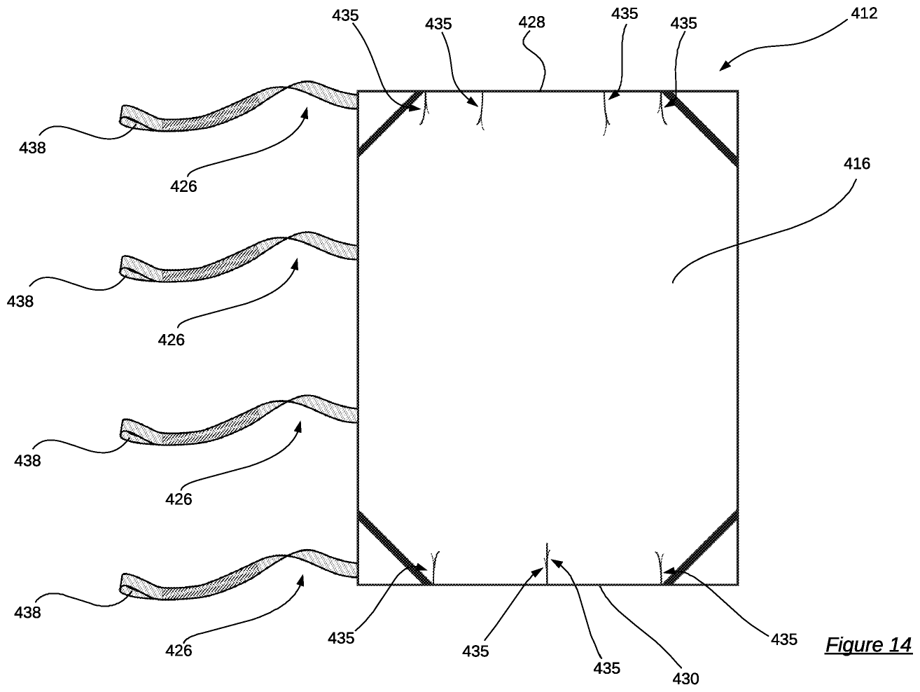

FIGS. 13 and 14 show an overlay 412 according to a fourth embodiment, the overlay 412 being for use in a corporeal compression system. The overlay 412 is substantially similar to the overlay 12 of the corporeal compression system 10 of FIG. 1. Accordingly, components of the overlay 412 that are similar to components of the overlay 12 have the same number with the prefix “4”.

The overlay 12 has flexible sheet material that is arranged into layers 416, 418. FIG. 13 shows the anterior side of the overlay 412, and thus the anterior layer 418. FIG. 14 shows the posterior side of the overlay 412, and thus the posterior layer 416.

An internal region (not shown in FIGS. 13 and 14) is defined between the layers 416, 418. As the sheet material of the layers 416, 418 is flexible, the layers can be repositioned with respect to each other. In a preferred embodiment, the flexible sheet material is a laminate of nylon and thermoplastic polyurethane. This material has the benefit of having low air flow rate through the material. In addition, the material can be joined by heat welding, which minimizes holes (and thus leaks) at the seams.

To facilitate movement of the posterior layer 416 during inflation, the portion of flexible sheet material that forms the posterior layer 416 is wider than the portion of flexible sheet material that forms the anterior layer 418. To accommodate the difference in width in the portions of flexible sheet material for the layers 416, 418, the posterior layer 416 is formed with pleats 435. Each pleat 435 extends in the length direction of the overlay 412, from one the superior edge 428 or inferior edges 430 of the overlay 412. In this particular embodiment, the overlay 412 has eight pleats 435; four pleats 435 being distributed approximately evenly along the superior edge 428, and four pleats 435 at the inferior edge 430. As shown in FIG. 14, the middle two of the four pleats 435 at the inferior edge 430 are beside one another at the centreline of the overlay 412.

The overlay 412 further has an inlet connector 422 within the anterior layer 418. The inlet connector 422 defines an inlet orifice that opens into the internal region. The pump 14 has an outlet pipe 24 that, in this particular embodiment, is releasably connectable to the inlet connector 22. In this way, gas from the pump 14 is delivered through the outlet pipe 24, and into the internal region 20.

The overlay 412 has four restraints 426 for restraining the overlay 412 to a support surface 5, such as a bed. In this embodiment, the restraints 426 are attached to the anterior layer 418. The length of each restraint 426 is sufficient to extend underneath, and thus around, the theatre bed and a patient, and overlap with itself.

Each restraint 426 has a releasable coupling that, in this embodiment is in the form of hook and loop fastener materials 434, 436. In this example, the hook material 434 is provided on a portion of the “free” section of the restraint 426. The loop material 436 is provided across the width of the flexible sheet material, and on the external surface of the anterior layer 418. Each restraint 426 includes a looped handle 438 at the terminal end of the respective restraint 426.

Corporeal compression systems in accordance with embodiments may find possible use in scenarios and fields, including (but not limited to):

treatment of distributive shock, including:
Sepsis, in short term assessment and/or prolonged treatment, where fluid volume replacement improves vascular status, and
Anaesthetic related hypotension;

placing of Central Venous Lines (CVL), whereby elevated venous pressure distends the patient's veins, making needling easier;

short term management of venous bleeding;

Central System investigations, including:
Cardiopulmonary Resuscitation (CPR), to increase cardiac preload;
Interposed Abdominal Pressure during CPR, by providing compression to the patient's entire body below and including the abdomen;
Dobutamine Stress echo to stop SAM, caused by redistribution of blood from the dobutamine venodilation), and
Pre-load stress the heart to assess diastolic heart failure;

transportation of trauma patients suffering:
intra-abdominal, such as an Abdominal Aortic Aneurysm (AM), pelvic injuries, including (pelvic fractures), or lower limb bleeding, and
Lower limb fractures;

and sports recovery and treatments.

One element common to all uses of the corporeal compression system is the need for a transient application of compression to the abdomen and lower limbs of a patient who is positioned on a supporting surface.

As described above, embodiments of the corporeal compression system can be used with a theatre bed and EMS stretchers. It will be appreciated that embodiments of the corporeal compression system can be used with many other objects that provide a supporting surface on which a person can be positioned. These include (but are not limited to) spinal boards, split-board stretchers, hospital trolleys, beds, and procedure specific patient beds.

It will be understood that values of pressure stated throughout this specification and claims are gauge pressure (and not absolute pressure), except where the context indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A corporeal compression system for use in applying compression to a patient positioned upon a supporting surface, the corporeal compression system comprising:
an overlay having:
flexible sheet material that is arranged into at least two layers to define an internal region therebetween, the layers being repositionable with respect to each other such that the sheet material can assume a deflated state in which the volume of the internal region is a minimum, and
an inlet orifice that opens into the internal region;
one or more restraints that, in use, locate and restrain edge portions of the flexible sheet material relative to the supporting surface with at least part of the patient's body between the supporting surface and the flexible sheet material; and
a gas supply system that has a discharge port that is connected to, or is connectable to, the inlet orifice, the gas supply system being configured to deliver gas through the discharge port,
wherein, in use of the corporeal compression system:
the flexible sheet material of the overlay is draped over the patient so as to provide a posterior layer that is in contact with the patient and an anterior layer that is spaced from the patient by the posterior layer,
the flexible sheet material is restrained relative to the support surface by the restraints, and
the gas supply system is operable to deliver gas via the inlet orifice to increase the volume of the internal region from the deflated state and establish an elevated pressure within the internal region, thereby compressing the patient between the posterior layer of the overlay and the supporting surface.

2. The corporeal compression system according to claim 1, wherein the gas supply system further comprises one or more of:
a containment vessel that is configured to contain pressurized gas, and a gas distribution circuit interconnects the containment vessel with the discharge port;
a pump having the discharge port through which to discharge gas; and
an inflow connector and gas distribution circuit, the inflow connector interconnecting the discharge port with an independent supply of pressurized gas via the gas distribution circuit,
wherein, in use of the corporeal compression system, the gas supply system is operable to deliver gas to the overlay at a first flow rate and at a second flow rate, wherein the first flow rate is higher than the second flow rate, and wherein the gas supply system is configured to deliver gas at the second flow rate when the pressure within the internal region is above atmospheric.

3. An overlay for use in applying compression to a patient positioned upon a supporting surface, the overlay comprising:
flexible sheet material that is arranged into at least two layers to define an internal region therebetween, the layers being repositionable with respect to each other such that the sheet material can assume a deflated state in which the volume of internal region is a minimum; and
an inlet orifice that opens into the internal region,
wherein, in use of the overlay:
the flexible sheet material in the deflated state is draped over the patient so as to provide a posterior layer that is in contact with the patient and an anterior layer that is spaced from the patient by the posterior layer,
the flexible sheet material is restrained relative to the support surface, and
gas is introduced to the internal region via the inlet orifice to increase the volume of the internal region from the deflated state and establish an elevated pressure within the internal region, thereby compressing the patient between the posterior layer of the overlay and the supporting surface.

4. The overlay according to claim 3, wherein in use a portion of the posterior layer conforms to the patient, and the anterior layer is distended when an elevated pressure is established within the internal region.

5. The overlay according to claim 3, wherein the overlay includes restraints with which to restrain the flexible sheet material to the supporting surface.

6.-7. (canceled)

8. The overlay according to claim 3, wherein the layers of the overlay include an inner layer that is to provide the posterior layer in use of the overlay, and an outer layer that is to provide the anterior layer in use of the overlay,
and wherein the inner layer includes one or more pleats that extend in the length direction of the overlay.

9.-10. (canceled)

11. The overlay according to claim 3, wherein the flexible sheet material includes a superior peripheral edge and an inferior peripheral edge,
whereby, in use of the overlay, the inferior peripheral edge is to be further from the patient's head than the superior peripheral edge.

12. (canceled)

13. The overlay according to claim 3, wherein the inlet orifice is part of an inlet connector, and the discharge port is part of an outflow connector that interconnects with the inlet connector.

14. A gas supply system for use with an overlay of a corporeal compression system, the gas supply system comprising:
a discharge port that is connected to, or is connectable to, an inlet orifice of the overlay, one or more of:
a pump that is in communication with the discharge port;
a containment vessel that is in communication with the discharge port via a gas distribution circuit, the containment vessel being configured to contain pressurized gas; and
a gas distribution circuit that includes an inflow connector that is to interconnect with an independent supply of pressurized gas, and one or more conduits that direct gas from the inflow connector to the discharge port;
wherein in use of the gas supply system within the corporeal compression system:
the gas supply system is operable to deliver gas to the overlay at a first flow rate and at a second flow rate, the first flow rate being higher than the second flow rate, and
the gas supply system is configured to deliver gas at flow rates up to the second flow rate when the pressure within the internal region is above a pre-determined pressure.

15. The gas supply system according to claim 14, wherein the pump includes:
an electric motor connected to a rotor that is rotatable to displace gas from an intake, through a chamber in which the rotor is housed, to the discharge port;
a discharge connector that interconnects the discharge port with a complementary connector that is in communication with the inlet orifice of the overlay;
at least one of a flow sensor and a pressure sensor located between the chamber and the discharge port;
a controller that controls the operation of the electric motor, the controller being configured to receive information from the flow sensor and/or the pressure sensor, and to drive the electric motor to vary the flow rate of gas to the discharge port in response to the received information; and
at least one of: a self-contained source of electrical power, and a connector with which to connect the pump to an independent source of electrical power, that provides electrical power to the electric motor.

16. The gas supply system according to claim 15, wherein the controller is configured such that the pump is operable at a first flow rate to inflate the overlay, and at flow rates up to a second flow rate to establish and/or maintain an elevated pressure within the internal region, the first flow rate being higher than the second flow rate.

17. The gas supply system according to claim 15, wherein the controller is configured so that when initialised, the controller is set to initially drive the electric motor to supply gas to the discharge port at the first flow rate.

18. The gas supply system according to claim 15, wherein the controller is configured so that when initialised, the controller drives the electric motor to supply gas to the discharge port at the first flow rate to discharge a pre-determined volume of gas, and thereafter drives the electric motor to supply gas to the discharge port at flow rates up to the second flow rate.

19. The gas supply system according to claim 15, wherein the controller is configured so that when the sensed pressure is at or below a pre-determined threshold pressure, the controller drives the electric motor to supply gas to the discharge port at the first flow rate to discharge a pre-determined volume of gas, and when the sensed pressure is above the pre-determined threshold pressure, the controller drives the electric motor to supply gas to the discharge port at flow rates up to the second flow rate.

20. The gas supply system according to claim 15, further comprising an input user interface that enables a user to set a pre-determined set point pressure.

21.-31. (canceled)

32. A corporeal compression system comprising:
a gas supply system comprising:
a discharge port forming an outflow connector;
a pump that is in communication with the discharge port, the pump including:
an electric motor connected to a rotor that is rotatable to displace gas from an intake, through a chamber in which the rotor is housed, to the discharge port,
at least one of a flow sensor and a pressure sensor located between the chamber and the discharge port,
a controller that controls operation of the electric motor, the controller being configured to receive information from the flow sensor and/or the pressure sensor, and to drive the electric motor to vary the flow rate of gas to the discharge port in response to the received information, and
at least one of: a self-contained source of electrical power, and a connector with which to connect the pump to an independent source of electrical power, that provides electrical power to the electric motor,
the gas supply system being operable to deliver gas to the discharge port at a first flow rate and at a second flow rate, the first flow rate being higher than the second flow rate;
an overlay for applying compression to a patient positioned upon a supporting surface, the overlay comprising:
flexible sheet material that is arranged into at least two layers to define an internal region therebetween, the layers being repositionable with respect to each other such that the sheet material can assume a deflated state in which the volume of internal region is a minimum,
an inlet orifice that opens into the internal region, and
a conduit that is interconnected at a first end to the flexible sheet material so as to open into the inlet orifice, the second end of the conduit including an inlet connector that is releasably couplable to the outflow connector,
wherein, in use of the corporeal compression system:
the flexible sheet material of the overlay, in the deflated state, is draped over the patient so as to provide a posterior layer that is in contact with the patient and an anterior layer that is spaced from the patient by the posterior layer,
the flexible sheet material is restrained relative to the support surface, and
the inlet connector is coupled to the outflow connector, whereby the coupling action operates the electrical switch, thereby operating the gas supply system to introduce gas to the internal region via the inlet orifice at the first flow rate to increase the volume of the internal region from the deflated state and establish an elevated pressure within the internal region, thereby compressing the patient between the posterior layer of the overlay and the supporting surface,
and wherein the gas supply system is configured to deliver gas at flow rates up to the second flow rate when the pressure within the internal region is above a pre-determined pressure.

33. The corporeal compression system according to claim 32, wherein the inlet connector and outflow connector are configured such that an elevated pressure within the conduit biases the inlet connector and outflow connector into the coupled state.

34. The corporeal compression system according to claim 32, wherein the pump includes a spring that is positioned to bias the inlet connector and outflow connector into the coupled state.

35. The corporeal compression system according to claim 32, wherein the inlet connector and outflow connector form a bayonet mount, and the electrical switch is positioned relative to the outflow connector so as to be actuated after the first movement of coupling the inlet connector and outflow connector is complete.

36. The corporeal compression system according to claim 35, wherein the electrical switch is positioned relative to the outflow connector so as to be actuated during the first movement of decoupling the inlet connector and outflow connector.
